(19) United States
(12) Patent Application Publication
(10) Pub. No.: US 2022/0313816 A1
(43) Pub. Date: Oct. 6, 2022

(54) COMPOSITIONS AND METHODS FOR ANTIBODY-AS-ADJUVANT VACCINES AND THERAPEUTICS

(71) Applicant: Human Vaccines Project, New York, NY (US)

(72) Inventors: Tobias KOLLMANN, New York, NY (US); Arnaud MARCHANT, New York, NY (US); Wayne KOFF, New York, NY (US)

(21) Appl. No.: 17/657,696

(22) Filed: Apr. 1, 2022

Related U.S. Application Data
(60) Provisional application No. 63/169,389, filed on Apr. 1, 2021.

(51) Int. Cl. A61K 39/39; A61P 37/04

(52) U.S. Cl. CPC A61K 39/39; A61P 37/04; A61K 2039/55511

(57) ABSTRACT

The invention provides antibody as adjuvant compositions and methods of using the same. Antibodies as adjuvant compositions can include antibodies that specifically bind to an antigen for use in a vaccine or can be non-specific toward the antigen.

### CROSS-REFERENCE TO RELATED APPLICATION

This application claims benefit of U.S. Provisional Patent Application No. 63/169,389, entitled “COMPOSITIONS AND METHODS FOR ANTIBODY-AS-ADJUVANT VACCINES AND THERAPEUTICS”, filed Apr. 1, 2021, the disclosures of which are hereby incorporated by reference in their entirety.

### FIELD OF THE INVENTION

The present invention relates to an adjuvant for an enhanced immune response in use with vaccine antigens and therapeutics, and more particularly, an antibody-as-adjuvant for targeted immune response for use with vaccine antigens and immune-based therapeutics.

### BACKGROUND OF THE INVENTION

Adjuvants facilitate protective immunity when included in some vaccines. In the past 90 plus years, adjuvants have been combined with antigens in hopes of producing a more robust immune response than with antigen alone. Numerous adjuvant compounds have been assessed over the years including: mineral salts, microbial products, emulsions, saponins, cytokines, polymers, microparticles, and liposomes (Guy B, The perfect mix: recent progress in adjuvant research. Nat Rev Microbiol 2007 Jul, 5(7) 505-517). Although a few adjuvants have proven helpful over the years, most have not. In the past twenty years, few new adjuvants, like MF59 and AS03, have been approved by the FDA, and only for specific antigen incorporation (seasonal influenza, avian influenza, and pandemic influenza). These adjuvants are both squalene-based, and specific in utility for influenza.

The need for improved adjuvants will continue as antigen components in vaccines are moving toward safer purified (and often recombinant) antigens. These purified recombinant antigens remove the need for vaccine manufacturers to use live attenuated virus or inactivated virus. However, purer components have tended to be lower in immunogenicity, in contrast to the live attenuated or inactivated whole-cell vaccines. Thus, there is a continued need for safe adjuvants that result in an enhanced immune response.

### SUMMARY OF THE INVENTION

Embodiments in accordance with the present invention include the use of antibody as adjuvants for inclusion with various antigens in vaccines. In some aspects the antibody is specific to the antigen found in the vaccine, i.e., the antigen specifically binds the antigen. For example, the antibody can be a monoclonal antibody raised against the vaccine antigen. In other aspects, the antigen is non-specific for binding to the antigen. It is also contemplated that the adjuvant be a combination of specific and non-specific antibody to the vaccine antigen, including radios of 50:50 specific to non-specific.

Antibody adjuvants can include other compounds (aluminum-based adjuvants for example) or can be administered alone. Antibodies can be of any isotype, and can include a combination of one or more isotypes in the same adjuvant. For example, an adjuvant in accordance with embodiments herein can be an antibody specific to the antigen and be IgG only, or could be an antibody specific to the antigen and be a mix of IgM, IgG, IgA, IgE and/or IgD isotypes.

Any of the embodiments and aspects described herein can be used in conjunction with one another, unless otherwise indicated or apparent from the context. Other embodiments will become apparent to those skilled in the art from a review of the ensuing detailed description. The following detailed description includes exemplary representations of various embodiments of the invention, which are not restrictive of the invention as claimed. The accompanying figures constitute a part of this specification and, together with the description, serve only to illustrate embodiments and not to limit the invention.

### DETAILED DESCRIPTION OF THE EMBODIMENT OF THE INVENTION

### Definitions

As used herein, the term “about” refers to an insignificant modification or variation of the numerical value that the basic function of the item to which the numerical value relates is unchanged by less than ±10%, and more typically less than ±5%.

As used herein, the terms “a” and “an” refer to one or to more than one of the grammatical objects of the article. By way of example, “an element” means one element or more than one element.

As used herein, “antibody” refers to a whole antibody molecule as well as to fragments of the antibody. Whole antibodies are composed of two different kinds of polypeptide chain, heavy (H) and light (L). Antibody herein includes the five isotypes, IgG, IgA, IgE, IgM, and IgD. Each IgG molecule, for example, has two heavy chains and two light chains, such that each heavy chain is linked to the other via a disulfide bond, and each light chain is linked to a heavy chain by a disulfide bond. Antibody herein also refers to antibody fragments, for example, Fab (antigen binding fragment), scFv (single chain variable fragment), VH (variable heavy chain) and VL (variable light chain) and Fab2. Antibody also includes monoclonal antibodies, polyclonal antibodies and genetically engineered antibodies.

As used herein, the term “antigen” refers to a virus, bacteria, parts of a virus or bacteria, a foreign or tumor (cancer) protein or nucleic acid that is to provide the target for the immune system. The immune system can be stimulated to elicit an immune response meant to target (e.g. attack and destroy or simply bind) the antigen.

As used herein, the term “biologic sample” refers to any sample from a subject, including fluids (whole blood, plasma, lymph, saliva, etc.) and tissue and tissue biopsies.

As used herein, the term “biomarker” refers to any detectable compound, such as a protein, peptide, proteoglycan, glycoprotein, nucleic acid, small molecule (metabolite), or fragments of any of the above. Biomarkers can be isolated from a biological sample, directly measured in the biological sample, or detected to be in a biological sample.

The phrase “immune response” refers to a response in an organism against foreign agents. An immune response is mediated by the action of a cell and immune system (for example, a T lymphocyte, B lymphocyte, natural killer cell, macrophage, eosinophil, mast cell, dendritic cell or neutrophil etc.) and soluble macromolecules produced by any of these cells or other host organs (e.g. the liver) that results in selective targeting, binding to, damage to, destruction of, and/or elimination from the organism's body of foreign agents which may include invading pathogens, cells or tissues infected by pathogens, or other like pathogenic targets.

As used herein, the term “individual,” “patient” and “subject” is used interchangeably, and refers to mammals, including, but not limited to, humans, other non-human primates (e.g. prosimians, monkeys, apes, etc), livestock (horses, cattle, goats, swine, etc), domestic pets (cats, dogs, etc), and the like.

As used herein, the term “reference value” or “control value” or control can be an absolute value, a relative value, a value with an upper and lower limit, or a range of values.

As used herein, “passive antibodies” refers to antibodies introduced into a subject without concomitant introduction into the organism of the B cells that made these particular antibodies. Passive antibodies do not require direct exposure of the subject to the particular antigen or pathogen.

As used herein, “adjuvant” refers to a composition or material used to enhance a subject's immune response to an antigen.

As used herein, “immunization” refers to the process whereby a subject is made immune or resistant to an infectious disease or produces antibodies or T cells specific for the inciting antigen or pathogen.

As used herein, “vaccine” refers to a product, the administration of which is intended to elicit an immune response that targets the specific antigen of interest. A vaccine antigen in accordance with embodiments herein can be a live attenuated preparation of bacteria, viruses or parasites, inactivated/killed whole organisms, living irradiated cells, crude fractions or purified antigens (sometimes then called ‘immunogens’). Vaccines can also include DNA, conjugates of various proteins, synthetic antigens, recombinant proteins, polynucleotides, plasmids, vectors, cells pulsed with immunogen, or any combination or like agent listed above.

Unless defined otherwise, all terms and phrases used herein include the meanings that the terms and phrases have attained in the art, unless the contrary is clearly indicated or clearly apparent from the context in which the term or phrase is used.

The present invention provides antibody-as-adjuvants and further, in some aspects antibodies with or without other known adjuvant ingredients; as well as methods described herein of using the same.

Embodiments herein are directed at inclusion of antibody-as-adjuvants for use in a vaccine. Embodiments include compositions having a predetermined amount of antibody in sterile water or PBS, for example. Antibody compositions can be the vaccine adjuvant alone, or can be combined with other known adjuvant ingredients for inclusion in the vaccine. Where used, base adjuvants for use with antibody compositions herein include, but are not limited to, aluminum-containing adjuvants (alum) having aluminum hydroxide, aluminum phosphate, amorphous aluminum hydroxyphosphate sulfate, or potassium sulfate, monophosphoryl lipid with aluminum salts (AS04), oil in water emulsions composed of squalene, monophosphoryl lipid A and QS-2lposomal formulation), ISCOMs containing adjuvants and cytosine phosphoguanine. As noted above, where the antibodies do not require inclusion of a base adjuvant, the antibodies would be delivered in a sterile water or saline or like liquid with a target antigen to compose the vaccine.

In various embodiments, the inventors generally provide antibody containing adjuvants for inclusion with target antigens. In some aspects, antibody-as-adjuvant can also be administered separately from antigen in time or space (for example injected in different muscles and/or at different times). In some aspects, the antibodies in the adjuvant are antigen specific. In other aspects, the antibodies are non-specific to the antigen. In some cases, the antibody, including antigen specific antibody, is IgG of any subclass and may have post-translational modifications (such as glycosylation, phosphorylation, methylation, acetylation, lipidation, and the like). In other cases, the antibodies in the adjuvant are antibody fragments having antigen specificity, or a combination of antibody and antibody fragments to a specific antigen. It is also envisioned that due to the wide range of antigens that can be targeted in a vaccine, a plurality of antibody and antibody fragments can be included in the adjuvant, each part of a group to various antigens of interest. It is also envisioned that the antibodies in use can be isotype IgM, IgE, IgA, IgG or IgD, or could include a mix of any two or more antibody isotypes.

In some aspects the amount of antibody to include in the adjuvant is appropriate for a single dose of vaccine. After combination of the antibody and/or antibody fragments in the adjuvant, the adjuvant can have its pH modified to an acceptable pH for injection into a subject. In some cases, the pH is modified by known pH modifying agents like HCl or NaOH. In some cases, the antibody containing adjuvant includes a salt like NaCl or other like salt.

In another embodiment, the amount of antibody above is combined with a single dose of known adjuvant. For example, an amount of antibody combined with an aluminum-containing adjuvant to prepare a single dose for inclusion in a vaccine. An amount of antibody combined with ISCOMs to prepare a single dose for inclusion in a vaccine. An amount of antibody combined with an oil in water emulsion to prepare a single dose for inclusion in a vaccine. An amount of antibody combined with squalene-based emulsions to prepare a single dose for inclusion in a vaccine. An amount of antibody combined with cytosine phosphoguanine to prepare a single dose for inclusion in a vaccine. Vaccines can be prepared for a single dose or multiple doses, and adjuvant and antigens can be combined and then stored, or stored separately and then combined at time of administration.

As such, vaccine compositions can comprise an antibody and antigen, or can consist of an antibody and an antigen. In some cases, the vaccine compositions can also consist essentially of an antibody and an antigen.

In some embodiments, a method for treating a subject in need of a vaccine is provided. The method includes mixing a dose of antibody and combining it with an antigen useful in the vaccination of the subject. In some aspects, a known adjuvant can be combined with the antibody and antigen. In other embodiments, the antibody is used without any other adjuvant. Antibody can be antigen specific or can be non-specific to the antigen in use. Antibody can be of any isotype, including IgG. Subjects can be pregnant women, newborns, kids under the age of two, kids between the ages of 2 and 10, young adults, immunocompromised individuals, and adults.

In some embodiments, a method for treating a subject in need of a vaccine is provided. The method includes identifying a subject in need of such administration; procuring a dose of antibodies against one or more antigens found in the vaccine, combining the dose of antibodies with the one or more antigens found in the vaccine to produce a single dose of vaccine; administering the vaccine to the subject and testing the subject after a predetermined amount of time for antiserum to the vaccine.

In some embodiments a method for treating a subject in need of a vaccine is provided. The method includes identifying a subject in need of such administration; procuring a dose of antibodies against one or more antigens found in the vaccine; combining the dose of antibodies with a dose of adjuvant; combining the dose of antibodies and adjuvant with the vaccine antigen to produce a single dose of vaccine; administering the vaccine to the subject and testing the subject after a predetermined amount of time for antiserum to the vaccine. In some aspects, the combining of the single dose of antibodies, adjuvant and antigen can be reversed or in any order that combines the three ingredients. In some aspects, the antibodies are IgG and the adjuvant is selected from an aluminum-containing adjuvant, an oil and water emulsion, monophosphoryl lipid A and QS-21, and cytosine phosphoguanine.

In other embodiments, methods for treating a pregnant woman in need of a vaccine are provided. The methods includes procuring a dose of antibody against one or more antigens found in a vaccine; combining the dose of antibodies with a dose of adjuvant; combining the dose of antibodies and adjuvant with the vaccine antigen to produce a single dose vaccine; administering the vaccine to the pregnant woman and testing the pregnant woman after the woman has given birth for antiserum to the vaccine. As above, the combining of the single dose of antibodies, adjuvant and antigen can be reversed or in any order that combines the three ingredients. In some cases, the baby is also tested upon birth for antiserum to the vaccine.

In still other embodiments, methods for treating a newborn baby in need of a vaccine are provided. The methods includes procuring a dose of antibody against one or more antigens found in a vaccine; obtaining a dose of adjuvant for combination with the antibodies; combining the dose of antibodies, vaccine antigen and adjuvant to form a single dose of vaccine; administering the single dose of vaccine to the newborn baby. In some aspects, the newborn baby can be tested for antiserum to the vaccine.

In each of the above methods, administering two, three or more doses of vaccine over the course of several weeks, months or years is also envisioned.

### EXAMPLES

The invention will be further illustrated by the following nonlimiting examples. These Examples are set forth to aid in the understanding of the invention but are not intended to, and should not be construed to, limit its scope in any way. The Examples do not include detailed descriptions of conventional methods that would be well known to those of ordinary skill in the art (molecular cloning techniques, cell culturing, biochemical assays, etc.).

Infectious morbidity and mortality is highest in the first weeks after birth. This vulnerability is not unexpected given the predominantly naYve state of neonatal immune cells and unique immunological challenges at birth, which require discrimination between not only innocuous self and non-inherited maternal antigens, but also an infinite array of foreign antigens associated with the shift to oral nutrient ingestion and primary commensal colonization (Basha, Surendran, Pichichero, Immune responses in neonates. Expert Rev Clin Immunol 10, 1171-1184 (2014)). This need for expanded immunological tolerance parallels skewed responsiveness amongst a variety of neonatal immune components, and accumulation of cells with suppressive properties (Elahi et al., Immunosuppressive CD71+ erythroid cells compromise neonatal host defense against infection. Nature 504, 158-162 (2013)).

Vaccination remains one of the most cost-effective ways for preventing infection. Vaccines against poliomyelitis, hepatitis B, tuberculosis, tetanus, pertussis, diphtheria, Haemophilus influenza type b (Hib), rotavirus and measles are not given to millions of infants, preventing an estimated 2.5 million deaths each year (MacLennan, Saul, Vaccines against poverty. Proc Natl Acad Sci USA 111, 12307-12312 (2014)). While vaccination has clearly benefited older infants and children, it has been considerably less effective in protecting against infection in the first month of life (Wang et al., Global, regional, and national levels of neonatal, infant and under-5 mortality during 1990-2013: a systematic analysis for the Global Burden of Disease Study 2013. Lancet 384, 957-979 (2014). The World Health Organization currently recommends administration of vaccines against tuberculosis, hepatitis B and polio as soon as possible after birth (<24 hours) to accelerate accumulation of protective immune components. Likewise, maternal vaccination against tetanus (Khan et al., Maternal and neonatal tetanus elimination: from protecting women and newborns to protecting all. Int J Womens Health 7, 171-180 (2015)), influenza virus (Madhi et al., Influenza vaccination of pregnant women and protection of their infants. N Engl J Med 371, 918-931 (2014)), and pertussis (Amirthalingam et al., Effectiveness of maternal pertussis vaccination in England: an observational study. Lancet 384, 1521-1528 (2014)) can efficiently protect through vertically transferred immunity and reducing infant pathogen exposure. However, vaccines currently in use do not target the most relevant neonatal pathogens especially in lower and middle income countries, where most severe newborn infection occurs. A recent meta-analysis of studies between 2008-2018 highlighted S. aureus, Klebsiella and E. coli spp. as the dominant causes of bacteremia/sepsis in neonates (<28 days) in sub-Saharan Africa (Okomo et al., Aetiology of invasive bacterial infection and antimicrobial resistance in neonates in sub-Sahara Africa: a systematic review and meta-analysis in line with the STROBE-NI reporting guidelines. Lancet Infect Dis 19, 1219-1234 (2019)). Urea plasma spp. and Group B streptococcus (GBS) were most frequently identified amongst cases of suspected early onset(ty 3 days) sepsis in South Africa (Velaphi et al., Surveillance for incidence and etiology of early-onset neonatal sepsis in Soweto, South Africa. PLoS One 14, e0214077 (2019)), whereas respiratory syncytial virus (RSV) and Ureaplasma spp. were the most common identified pathogens in cases of possible serious bacterial infection in <60 day old infants in south Asia (Saha et al., Causes and incidence of community-acquired serious infections among young children in south Asia (ANISA): an observation cohort study. Lancet 392, 145-159 (2018)). Although study-dependent differences in microbial detection methods and age of subjects analyzed in each geographical region preclude precise estimates of combined global disease burden for individual pathogens, clearly emerging is that presently used vaccines are not targeting the most relevant neonatal pathogens (see FIG. 2). Thus, alternative strategies as described herein are desperately needed. In the following examples, we summarize the principles underpinning vaccination of newborns, their mothers, and importantly, the mother-newborn dyad as one immunological unit to enhance early life immunity.

### Example 1: Pathogen-Specific Immunity after Newborn Vaccination

The human newborn is often inappropriately considered immature or prone to immunological tolerance, and therefore unable to respond to vaccination. This misconception likely arose after extrapolating results obtained in narrowly focused studies to the entire newborn immune system, such as induction of tolerance to allogenic tissue in newborn mice (Billingham, Brent, Medawar, Actively acquired tolerance of foreign cells. Nature 172, 603-606 (1950), and reduced responsiveness of human newborns to particular vaccines (Sauer, Immunization in early childhood. Ill Med J 97, 73-76, Illust (1950). For example, dampened antibody response to T cell independent polysaccharide antigens of encapsulated bacterial pathogens, including Haemophilus influenza type b (Hib) and Pneumococcus, until 2 years of age correlates with reduced marginal zone B cells (Klouwenberg, Bont, Neonatal and infantile immune response to encapsulated bacteria and conjugate vaccines. Clin Dev Immunol 2008, 628963 (2008)). Fortunately, conjugation of polysaccharide antigens to protein carriers allows T cells to be simultaneously activated, and robust protective antibody responses against these pathogens to be primed even in newborns (Kurikka et al., Neonatal immunization: response to Haemophilus influenza type b-tetanus toxoid conjugate vaccine. Pediatrics 95, 815-822 (1995)). Similarly, diphtheria-tetanus-whole cell pertussis (DTwP) and some acellular pertussis (DTaP) vaccine formulations were shown to elicit reduced responses in newborns compared with older infants (Sauer Id.). However, many studies have now demonstrated that monovalent acellular pertussis vaccines administered to newborns induce strong primary responses, and also do not induce tolerance to vaccine boosters (Saso, Kampmann, Vaccines responses in newborns. Semin Immunopathol 39, 627-642 (2017)) (FIG. 1). Vaccines administered in early life can also sometimes prime qualitatively different T cell responses. However, since the adaptive immune mediators responsible for protection have not been established for most vaccines, the clinical relevance of these qualitative differences remains difficult to interpret.

Newborns compared with older infants are quantitatively just as, if not more, responsive to vaccines currently included in the Expanded Programme of Immunization, namely bacilli Calmette-Guerin (BCG), oral polio vaccine (OPV) and Hepatitis B. The serological response of newborns is also comparable to infants for most other inactivated vaccines currently not licensed for neonatal administration such as rotavirus, monovalent pertussis, diphtheria or tetanus toxoid, and conjugated vaccines against Hib and Pneurnococcus (Saso Id., Wood et al., Immunogenicity and Safety of Monovalent Acellular Pertussis Vaccine at Birth: A Randomized Clinical Trial. JAMA Pediatr 172, 1045-1052 (2018)). Ample data further show vaccines, including live vaccines such as BCG have an outstanding safety record in newborns. Disseminated BCG infection is exceptionally rare and almost exclusively occurs in infants with underlying immune deficiency (Grange, Complications of bacilli Calmette-Guerin (BCG) vaccination and immunotherapy and their management. Commun Dis Public Health 1, 84-88 (1998)). Likewise, vaccine-associated polio (VAP) primarily occurs in under immunized populations which facilitate person-to-person spread, persistence and eventual reversion into a more virulent phenotype, and is expected to decline with reformulation of OPV (Platt, Estivariz, Sutter, Vaccine-associated paralytic poliomyelitis: a review of the epidemiology and estimation of the global burden. J Infect Dis 210 Suppl 1. S380-389 (2014)). Thus, newborns are very much capable of responding robustly and safely to most vaccines.

Given that newborns are capable of a robust vaccine response, why have current vaccination programs not lead to proportional mortality reductions in newborns as compared with older infants and children? Firstly, current vaccines administered to newborns do not specifically target pathogens that cause severe infection in the first weeks of life. Even though tuberculosis, hepatitis B or polio can be acquired by the newborn, these infections clinically manifest mostly outside the neonatal period. For pathogens that cause severe infection in the first weeks after birth, such as RSV, Ureaplasma app. and several other bacteria, vaccines are either not available or have not yet been tested in this neonatal window of vulnerability (FIG. 2). Secondly, priming a protective adaptive immune response in the immunologically largely naYve newborn commonly takes weeks (Hong, Infectious Diseases of the Fetus and Newborn Infant, Y. M. Christopher B Wilson, Jack S Remington, Jerome O Klein, Ed. (2016), pp 81-188), whereas infections often cause morbidity and mortality in the first few days after birth (Oza, Lawn, Hogan, Mathers, Cousens, Neonatal cause-of-death estimates for the early and late neonatal periods for 194 countries: 200-2013. Bull World Health Organ 93, 19-28 (2015), Saha et al., Causes and incidence of community-acquired serious infections among young children in south Asia (ANISA): an observation cohort study. Lancet 392, 145-159 (2018)). Delayed immune responsiveness in the newborn likely also explains their susceptibility to severe infection outcomes. This discordance between when infections occur, and the time it takes to prime pathogen-specific neonatal immune components makes vaccination strategies targeting newborn pathogens challenging. To more effectively protect infections manifesting in the newborn period, alternative approaches, such as promoting transfer of pathogen-specific maternal antibody through immunization during pregnancy and/or broadly increasing newborn resistance through pathogen-agnostic approaches, have to be considered.

### Example 2: Pathogen-Agnostic Protection after Newborn Vaccination

Non-pathogen-specific strategies known to reduce severe infection in newborns include breastfeeding (Victora et al., Breastfeeding in the 21st century, epidemiology, mechanisms, and lifelong effect. Lancet 387, 475-490 (2016)) and probiotics (Panigrahi et al., A randomized symbiotic trial to prevent sepsis among infants in rural India. Nature 548, 407-412 (2017)). Live vaccines can also broadly enhance neonatal host resilience beyond the vaccine's original pathogen target (L. C. J de Bree et al., Non-specific effects of vaccines: current evidence and potential implications. Semin Immunol 39, 35-43 (2018)). For example, BCG has long been recognized to stimulate a range of host responses beyond protection against tuberculosis. More recently meta-analysis encompassing>6,000 low-birth weight newborns show a 38% reduction in neonatal mortality attributed to BCG administered at birth (S. Biering-Sorensen et al., Early BCG-Denmark and Neonatal Mortality Among Infants Weighing <2500 g: A Randomized Controlled Trial. Clin Infect Dis 65, 1183-1190 (2017)). A separate study of over 7,000 normal birth weight newborns showed 40% reduced mortality when OPV was administered with BCG in the first 2 days of life (Lund et al., The effect of oral polio vaccine at birth on infant mortality: a randomized trial. Clin Infect Dis 61, 1504-1511 (2015)). These pathogen-agnostic protective effects appear to be fast-acting since significant reduction in overall mortality is identified in newborns already within the first 3 days after BCG administration, which stands in contrast to the weeks required to achieve pathogen-specific immunity (Hong, in Infectious Diseases of the Fetus and Newborn Infant, Y. M. Christopher B Wilson, Jack S Remington, Jerome O Klein, Ed. (2016). pp 81-188.). Enhanced serological responsiveness to other vaccines in infants administered BCG at birth further highlights broad immune stimulatory effects (Ota et al., Influence of Mycobacterium bovis bacillus Calmette-Guerin on antibody and cytokine responses to human neonatal vaccination. J Immunol 168, 919-925 (2002)).

The mechanism whereby live vaccines confer pathogen-agnostic protective effects have not been established but could involve cross-reactive T cells or enhanced protection via innate immunity (Goodridge et al., Harnessing the beneficial heterologous effects of vaccination.

Nat Rev Immunol 16, 392-400 (2016)). Another unresolved question is whether pathogen-agnostic protective effects primed by live vaccines are restricted to the neonatal period. Analysis of over 15,000 children in rural Guinea-Bissau showed mortality reductions associated with BCG vaccine scarring was restricted to those administered BCG within the first 4 weeks of life, and most pronounced amongst those vaccinated within the first week after birth (L Storgaard et al., Development of BCG Sear and subsequent morbidity and mortality in rural Guenea-Bissau. Clin Infect Dis 61, 950-959 (2015)). However, an expanded window of plasticity associated with pathogen-agnostic protective benefits is supported by similar reductions in childhood mortality associated with live attenuated measles vaccine administered after 4 months of age (P. Aaby et al., Reduced childhood mortality after standard measles vaccination at 4-8 months compared with 9-11 months of age. BMJ 307, 1308-1311 (1993)). Since pathogen-agnostic approaches have the potential to confer protection to the newborn that is broad and fast, thereby bypassing each of the drawbacks associated with current pathogen-specific strategies for newborn immunization, establishing the molecular and cellular mediators of protection are important next steps to further improve upon such approaches.

### Example 3: Pathogen-Specific Immunity after Vaccinating Mothers

Multiple immunological adaptations occur during pregnancy to accommodate growth and avert rejection of the semi-allogeneic fetus (A Erlebacher, Mechanisms of T cell tolerance towards the allogeneic fetus. Nat Rev Immunol 13, 23-33 (2013)). These tolerogenic adaptations are either anatomically confined and/or restricted to cells with fetal-specificity since the response to vaccines administered during pregnancy is largely comparable to that of non-pregnant women (Marchant et al., Maternal immunization: collaborating with mother nature. Lancet Infect Dis 17, e197-e208 (2017)). Vertically transferred maternal antibodies protect offspring in the early postnatal period. An important distinction compared with neonatal immunization is the transient nature of these protective benefits conferred to non-self-renewing antibodies that functionally persist in infants only for several months, thereby deferring infection to an age when the consequences are less severe.

Vaccination during pregnancy has already been shown to be effective for a few important neonatal infections. Mortality from neonatal tetanus is reduced by >90% after tetanus toxoid vaccination of pregnant women, and world-wide implementation could effectively eliminate this problem (H Blencowe et al., Tetanus toxoid immunization to reduce mortality from neonatal tetanus. Int J Epidemiol 39 Suppl 1, i102-109 (2010)). Protection of infants against respiratory illness and confirmed cases of influenza infection ranges from 30-60% when mothers are administered inactivated influenza vaccine during pregnancy (K Zaman et al., Effectiveness of maternal influenza immunization in mothers and infants. N Engl J Med 359, 1555-1564 (2008)), whereas protective efficacy against pertussis in the first 2-3 months of life is −90% following maternal vaccination (Amirthalingam et al., Effectiveness of maternal pertussis vaccination in England: an observational study. Lancet 384, 1521-1528 (2014)). With these benefits, an important aspect of these embodiments is developing vaccines for pregnant women targeting other neonatal pathogens such as RSV and GBS (P. T. Heath et al., Group B streptococcus and respiratory syncytial virus immunization during pregnancy: a landscape analysis. Lancet Infect Dis 17, e223-e234 (2017)).

Maternal antibodies vertically transferred across the placenta are almost exclusively IgG which exponentially increase in fetal tissues during the final weeks of gestation. Transfer is coordinated by maternal IgG binding to Fe receptors expressed by fetal trophoblasts, macrophages and endothelial cells (M. F. Jennewein et al., Transfer of maternal immunity and programming of the newborn immune system. Semin Immunopathol 39, 605-613 (2017)). Recent studies highlight the importance of specific Fc-glycan modifications that facilitate the selective transfer of IgG subsets efficient in antibody-dependent activation of neonatal innate NK cells (M. F. Jennewein et al., Fe Glycan-Mediated Regulation of Placental Antibody Transfer. Cell 178, 202-215 e214 (2019)). At term, IgG levels in the cord blood often exceed those in maternal blood further highlighting their active, instead of passive, transfer (N. E. Simister, Placental transport of immunoglobulin G. Vaccine 21, 3365-3369 (2003)). However, the temporal accumulation of maternal antibody in later gestation means that immunity primed by maternal vaccination is drastically different from preterm infants (C. Papadatos et al., Serum immunoglobulin G levels in small-for-dates newborn babies. Arch Dis Child 45, 570-572 (1970)). Likewise, IgG levels are also reduced amongst small for gestational age infants, and infants born to mothers with chronic inflammatory conditions including HIV infection or placental malaria (G. G. Pauda et al., The impact of IgG transplacental transfer on early life immunity. Immunohorizons 2, 14-25 (2018)).

Maternal IgA and IgG antibodies are also transferred to the newborn through breastfeeding, and increased levels of both subclasses can be detected in breastmilk following vaccination during pregnancy (Marchant et al., Id.). Optimization of newborn protection requires establishing the molecular determinants maternal antibodies transferred through breastmilk, and whether they can functionally complement placentally transferred antibodies.

Vaccinations during pregnancy raises understandable concerns with regards to safety. One consideration is vaccine-induced inflammation, and the potential impacts on the outcome of pregnancy. However, vaccines administered to pregnant women have excellent safety profiles, and there is no evidence of increased pregnancy complications with inactivated vaccines, adjuvanted with alum or oil-based emulsions (B Keller-Stanislawski et al., Safety of immunization during pregnancy: a review of the evidence of selected inactivated and live attenuated vaccines. Vaccine 32, 7057-7064 (2014)). Live attenuated vaccines are currently not recommended during pregnancy due to safety concerns. However, analysis following inadvertent administration suggest that live vaccines are also remarkably safe during pregnancy. For example, despite inadvertent administration of live attenuated rubella virus vaccine to >3,500 pregnant women with documented serological susceptibility, no cases of congenital rubella syndrome have ever been described, and only one case of infant asymptomatic virus shedding (J. Hofmann et al., Persistent fetal rubella vaccine virus infection following inadvertent vaccination during early pregnancy. J Med Virol 61, 155-158 (2000)). Likewise, mass administration of OPV or live attenuated yellow-fever vaccine in outbreak settings show no increased rates of growth retardation, congenital anomalies, or pregnancy complications in women vaccinated during pregnancy. One potential exception is smallpox vaccination. Even in this case, the largest current meta-analysis including over 12,000 pregnant women, showed only marginally increased incidence of congenital defects, with similar incidence of other complications including spontaneous abortion, stillbirth, preterm birth (M. L. Badell et al., Risks associated with smallpox vaccination in pregnancy: a systematic review and meta-analysis. Obster Gynecol 125, 1439-1451 (2015)). Collectively, these data indicate that even live vaccines are generally safe during pregnancy, especially when taking into account their proven protective benefits.

### Example 4: Linking the Maternal-Newborn Dyad

Chronic maternal infection with a variety of pathogens including helminths, HIV, and malaria, can impact infant health independently from pathogen transmission (Dauby et al., Uninfected but not unaffected: chronic maternal infections during pregnancy, fetal immunity, and susceptibility to postnatal infections. Lancet Infect Dis 12, 330-340 (2012)), along with the tempo and quality of immune development (Goetghebuer et al., Initiation of antiretroviral therapy before pregnancy reduces the risk of infection-related hospitalization in human immunodeficiency virus-exposed uninfected infants born in a high-income country. Clin Infect Dis 68, 1193-1203)). For example, HIV-exposed, but uninfected newborns have reduced levels of maternal antibodies and show susceptibility to severe infection by unrelated pathogens as compared with HIV-unexposed infants (Evans et al., HIV-exposed, uninfected infants: new global challenges in the era of pediatric HIV elimination. Lancet Infect Dis 16, e92-e107 (2016)). Cord blood cells from infants born to mothers with chronic HBV infection show increased production of some antimicrobial cytokines after stimulation with various bacterial pathogens (Hong et al., Trained immunity in newborn infants of HBV-infected mothers. Nat Commun 6, 6588 (2015)). Thus, in utero exposure to maternal infection has the potential to influence, both positively and negatively, the susceptibility of newborns to severe infection, reflecting an intimate link of immune fitness, defined as host resistance to severe infection, in the maternal-newborn dyad. The precise molecular and cellular mechanism whereby maternal infection and the prior immunological experience of mothers influences immune fitness of the newborn remain undefined, but likely involve in utero priming of fetal T and B lymphocytes, along with innate immune cells and/or shifts in the quantity-quality of maternal antibodies (Jennewein et al., Transfer of maternal immunity and programming of the newborn immune system. Semin Immunopathol 39, 605-613 (2017)).

Vertical transfer of antibodies from mother to offspring is teleologically conserved, and clearly provides immunity against infection in the newborn period (Chucri et al., A review of immune transfer by the placenta. J Reprod Immunol 87, 14-20 (2008)). As outlined above, maternal antibodies have unique biophysical characteristics as a result of pregnancy-induced modifications as well as selective transfer across the placenta that promote antimicrobial activity and interactions with innate immune cells in the newborn (Jennewein Id.). On the other hand, vertically transferred maternal immunity can negatively or positively influence immunity in newborns and infants primed by vaccines. For example, high titer maternal antibodies have often been associated with reduction of the primary antibody response of infants to vaccines (Niewiesk, Maternal antibodies: clinical significance, mechanism of interference with immune responses, and possible vaccination strategies. Front Immunol 5, 446 (2014)). A classical study prompted by increased incidence of symptomatic infection during measles outbreaks amongst children immunized before their first birthday showed a muted serological response to measles vaccination amongst children with high-titer pre-vaccine titers (Albrecht et al., Persistence of maternal antibody in infants beyond 12 months: mechanism of measles vaccine failure. J Pediatr 91, 715-718 (1977)). These findings have since been replicated in several other larger prospective cohorts, and for other live attenuated vaccines such as polio, influenza, varicella, rotavirus (Albrecht Id.). Interference by maternal antibodies has also been shown for infant serological responses to inactivated vaccines against tetanus, pertussis, pneumococcus, hepatitis A, and Hib where fold-increase in antibody titers after immunization consistently shows an inverse correlation with the levels of maternal antibody at birth (Jones et al., The relationship between concentration of specific antibodies at birth and subsequent responses to primary immunization. Vaccine 32, 996-1002 (2014)).

Interference by pre-existing antibody is not unique to infants, and instead likely reflects control of excessive antibody production classically described in adults (Uhr, Moller, Regulatory effect of antibody on the immune response. Adv Immunol 8, 81-127 (1968)). Masking of immunodominant epitopes, regulation of B cell activation and germinal center maturation, as well as B cell inhibition through FcrRlIB cross-linking are potential mechanisms (Vono et al., Maternal antibodies inhibit neonatal and infant responses to vaccination by shaping the early-life B cell repertoire within germinal centers. Cell Rep 28, 1773-1784 el 775 (2019)). Importantly, the priming of memory B cells is much less sensitive to the presence of high titers of pre-existing antibodies, since infant responses to vaccine boosters are consistently preserved with primary vaccination under the cover of high titers of maternal antibodies (Maertens et al., Pertussis vaccination during pregnancy in Belgium: Follow-up of infants until 1 month after the fourth infant pertussis vaccination at 15 months of age. Vaccine 34, 3613-3619 (2016)). T cell priming is also intact, since the presence of antibodies impacts neither proliferation nor effector cytokine production (Gans et al., IL-12, IFN-gamma, and T cell proliferation to measles in immunized infants. J Immunol 162, 5569-5575 (1999)). Robust memory B and T cell responses primed by vaccination of newborns under the cover of maternal immunity may be central to the success of vaccines that are routinely administered to newborn babies. Thus, interference by maternal antibodies is generally restricted to the primary serological response of offspring to vaccination, but the clinical implications on infection susceptibility remain uncertain.

In fact, vaccination of infants under the cover of maternal immunity may prime vaccine responses that are more protective, especially considering the aforementioned non-pathogen-specific protective benefits of live vaccines. For example, a striking 78% reduction in mortality (through 5 years of age) was shown for infants administered live attenuated measles vaccine at 4.5 months of age in the presence of maternal antibody compared with those without detectable maternal measles antibody at the time of vaccination (Aaby et al., Id). More recently, by stratifying infants born to mothers with a BCG scar indicating prior maternal priming, reduced infant mortality associated with BCG vaccination in the newborn period appears more pronounced in infants born to mothers with a BCG scar (Berendsen et al., Maternal priming: bacillus Calmette-Guerin (BCG) vaccine scarring in mothers enhances the survival of their child with a BCG vaccine scar. J Pediatric Infect Dis Soc, (2019)). A more balanced humoral and cellular response by vertically transferred factors including antibodies, cytokines, cells or metabolites likely explains these remarkable protective benefits. Inducing pathogen-specific along with pathogen-agnostic immunity by newborn immunization under the cover of maternal immunity has the potential to close the window of susceptibility to a large range of pathogens and to prime long-lived immunity (FIG. 3).

The necessity for individual cells and molecules in sustaining maternal-fetal tolerance have almost exclusively been established using preclinical pregnancy models (rodents) that do not recapitulate the more prolonged gestational length and in utero accumulation of fetal adaptive immune components in humans (Mold, McCune, Immunological tolerance during fetal development: from mouse to man. Adv Immunol 115, 73-111 (2012). Thus, establishing the mechanism for how vaccines targeting the maternal-newborn dyad work has exciting potential to simultaneously foster new approaches for averting equally daunting public health problems related to the maternal-fetal dyad such as preterm birth and stillbirth.

Vaccines that prime pathogen-specific immunity clearly work. As a result, we now are on the brink of eradicating poliomyelitis with vaccines administered in newborns, and eliminating once devastating infections of neonatal tetanus and congenital rubella are also within reach due to maternal and preconceptual vaccination. Boosted pathogen-agnostic immunity in newborns primed by live vaccines also shows promise, with up to 25-50% reductions in overall infant mortality. These successes should clearly remove misconceptions regarding the maturity of neonatal immune cells, safety of live vaccines and responsiveness of maternal immune components during pregnancy. In fact, combining these approaches by priming immunity in mothers followed by boosting newborns with vaccination under the cover of maternal immunity promises to simultaneously stimulate pathogen-agonistic as well as pathogen-specific immunity (see FIG. 3). Physicians are instructed first and foremost to do no harm. This instils a reflexive reluctance to deviate from the status quo. However, the current status quo is that nearly half of all under age 5 mortality occurs in neonates, and a large fraction of these are due to infection.

### EQUIVALENTS

Those skilled in the art will recognize or be able to ascertain using no more than routine experimentation, many equivalents of the specific embodiments of the invention described herein. Such equivalents are intended to be encompassed by the following claims.

Entire contents of all non-patent documents, patent applications and patents cited throughout this application are incorporated by reference herein in their entirety.

What is claimed is:

1. A method of inducing an immune response in a subject, comprising administering to the subject a vaccine, wherein the vaccine comprises an adjuvant and an antigen such that the adjuvant comprises antibodies specific for the antigen.

2. The vaccine of claim 1, wherein the antigen provides an immune response against measles, mumps, rubella, polio, varicella, tetanus/diptheria, hepatitis A, hepatitis B, Haemophilus influenzae B, whooping cough, pneumonia, influenza, Lyme's Disease, rotavirus, cholera, yellow fever, Japanese encephalitis, poliomyelitis, rabies, typhoid fever, pertussis, anthrax, Human Immunodeficiency Virus (HIV-1 and HIV-2), Avian Flu antigen, tumor antigens, Severe Acute Respiratory Syndrome, and tuberculosis (TB).

3. The vaccine of claim 1, wherein the antigen provides an immune response against cancer cells or prions.

4. The vaccine of claim 1, wherein the antigen provides an immune response against bacteria.

5. The vaccine of claim 1, wherein the antigen provides an immune response against viruses.

6. The vaccine of claim 1, wherein the antigen provides an immune response against a parasite.

7. The vaccine of claim 1, wherein the antibodies specific for the antigen are an IgG isotype.

8. The vaccine of claim 1, wherein the antibodies specific for the antigen can be of different isotypes.

9. A vaccine for immunizing a subject for a condition, the vaccine comprising: an antigen appropriate for generating an immunity in the subject for the condition; and an adjuvant comprising a predetermined amount of antibody.

10. The vaccine of claim 8, wherein the adjuvant only has the antibody, water or PBS, and optionally a salt and/or pH modifier.

11. The vaccine of claim 9, wherein the antibody is IgG isotype.

12. The vaccine of claim 9, wherein the antibody includes at least two or more isotypes.

13. The vaccine of claim 9, wherein the antibody is a monoclonal antibody that binds to the antigen.

14. The vaccine of claim 9, wherein the antibody is not specific to the antigen.

15. A kit comprising:
a single dose of antigen for an immune response;
a single dose of antibody for use as an adjuvant; and
one or more vials and/or syringes for mixing and administering the antigen and antibody.

15. The kit of claim 14, wherein the antibody is a monoclonal antibody that specifically binds to the antigen.

16. The kit of claim 14, wherein the antibody is non-specific to binding to the antigen.

17. The kit of claim 14, wherein the antigen is selected to immunize against mumps, measles, influenza, pertussis, hepatitis B, or polio.
